(19) United States
(12) Patent Application Publication
(10) Pub. No.: US 2016/0175618 A1
(43) Pub. Date: Jun. 23, 2016

(54) ULTRASONIC TREATMENT DEVICE

(71) Applicant: Chongqing Derma Optic&Electronic Technique Co., Ltd., Chongqing (CN)

(72) Inventor: Xiaobing LEI, Chongqing (CN)

(73) Assignee: Chongqing Derma Optic&Electronic Technique Co., Ltd., Chongqing (CN)

(21) Appl. No.: 15/055,609

(22) Filed: Feb. 28, 2016

Related U.S. Application Data
(63) Continuation of application No. PCT/CN2015/096098, filed on Dec. 1, 2015.

(30) Foreign Application Priority Data
Dec. 19, 2014 (CN) 201410793897.8
Dec. 19, 2014 (CN) 201410794091.0

(51) Int. Cl. A61N 7/00

(52) U.S. Cl. CPC A61N 7/00; A61N 2007/0091; A61N 2007/0034

(57) ABSTRACT

An ultrasonic treatment device includes a treatment main body, a treatment handpiece and a display control console, wherein the treatment main body is fittingly connected to the treatment handpiece; the display control console is coupled to the treatment main body via an interactive connection. A treatment handpiece is also provided for use with an ultrasound treatment device.

### CROSS-REFERENCE TO RELATED APPLICATIONS

The present application is a continuation of, and claims priority to, PCT/CN2015/096098, filed on Dec. 1, 2015, which claims priority to Chinese Patent Application No. 201410794091.0 filed on Dec. 19, 2014, and Chinese Patent Application No. 201410793897.8 filed on Dec. 19, 2014. The disclosures of these applications are hereby incorporated by reference in their entirety.

### BACKGROUND

Beauty-related products and devices have a huge market capacity. However, due to the high threshold of the medical and cosmetic industry, cosmetic products have been very expensive.

To date the industry of home-use medical devices has been in fast development largely due to the high market prospect of this industry. Home-use medical devices includes the detection devices and treatment devices, etc.

### SUMMARY

This disclosure relates generally to the field of medical equipment, and more specifically to a home-use medical device employing focused ultrasound wave as the energy source, and in more particular to an ultrasonic treatment device. It further specifically relates to a vagina treatment device which applies focused ultrasound, and in particular to an ultrasound vagina treatment handpiece.

Disclosed herein provides an ultrasonic treatment device, comprising a treatment main body, a treatment handpiece and a display control console, wherein: the treatment main body is fittingly connected to the treatment handpiece; the display control console is coupled to the treatment main body via an interactive connection; the treatment handpiece comprises a handle and a treatment tip, wherein the handle and the treatment tip are connected via a detachable connection, a transducer for focusing ultrasound is arranged in the treatment tip, and a motor for driving the transducer to move is arranged in the handle; the treatment main body comprises a driving unit, a control unit and an ultrasound power unit, wherein the driving unit is coupled to the motor in the handle, the ultrasound power unit is coupled to the transducer in the treatment tip, and the control unit is respectively coupled to the driving unit and the ultrasound power unit.

Preferably, in the above mentioned ultrasound treatment device, the interactive connection via which the display control console is coupled to the treatment main body is a wireless connection.

Preferably, in the above mentioned ultrasound treatment device, the display control console is a portable mobile device.

Preferably, in the above mentioned ultrasound treatment device, the portable mobile device is a tablet PC or a smart mobile phone.

Preferably, in the above mentioned ultrasound treatment device, the detachable connection via which the handle and the treatment tip are connected is through a card slot or a turning knob.

Preferably, in the above mentioned ultrasound treatment device, the transducer in the treatment tip is fittingly connected to the motor in the handle, and the motor is configured to move the transducer along a vertical direction or a horizontal direction.

Preferably, in the above mentioned ultrasound treatment device, an ultrasound coupling unit configured for adjusting treatment depth is arranged on a treatment side of the treatment tip.

Preferably, in the above mentioned ultrasound treatment device, treatment depth of the treatment tip is about 1 mm-5 mm.

Further, when the treatment depth reaches 10 mm-20 mm, the above mentioned ultrasound treatment device can be used to reduce the number of adipocytes.

The ultrasonic treatment device as disclosed herein has the following advantages. It is a home-use product applying focused ultrasound as energy source, which can fill in the niche of home-use tightening products in the market, and can greatly reduce customer's cost in cosmetics products. Meanwhile, the device can be controlled by a smart mobile phone or a tablet PC, further lowering the cost of this home-use device.

Disclosed herein also includes a treatment handpiece in an ultrasound treatment device according to some embodiments disclosed herein, which is specifically designed for use in a vagina ultrasound treatment device. It can be used to resolve the various problems of existing technologies, such as low treatment efficiency, fatigue symptoms of treated patients, as well as harming to the skin and the risk of infection during and after existing laser treatment processes.

This disclosure provides the following technical schemes:

Scheme 1

A treatment handpiece for use in an ultrasound vagina treatment device, comprising: a handle; a treatment tip, wherein the treatment tip comprises a lumen, is connected to the handle, and is of a cylindrical shape; a motor, wherein the motor is embedded in the handle, and a power-output shaft of the motor is arranged in the lumen of the treatment tip; a transducer for focusing ultrasound, wherein the transducer is placed inside the lumen of the treatment tip, and an annular spherical surface of the transducer is directly faced against a wall of the lumen of the treatment tip; a joining beam, wherein two ends of the joining beam are fittingly connected to the power-output shaft of the motor and the transducer respectively, and the transducer is configured to be driven by the motor to rotate in a direction perpendicular to an axial direction of the joining beam.

Preferably, the motor drives the transducer to rotate at an angle ranging from 3° to 30°.

Scheme 2

A treatment handpiece for use in an ultrasound vagina treatment device, comprising: a handle; a treatment tip, wherein the treatment tip comprises a lumen, is connected to the handle, and is of a cylindrical shape; a motor, wherein the motor is embedded in the handle, and a power-output shaft of the motor is arranged in the lumen of the treatment tip; a transducer for focusing ultrasound, wherein the transducer is placed inside the lumen of the treatment tip and is connected with the power-output shaft of the motor, an annular spherical surface of the transducer is directly faced against a wall of the lumen of the treatment tip; and the transducer is configured to be driven by the motor to move back and forth along an axial direction of the treatment tip.

Preferably, a linear distance of the transducer configured to move back and forth along the axial direction of the treatment tip is 0.5-5 mm.

Scheme 3

A treatment handpiece for use in ultrasound vagina treatment device, comprising: a handle; a treatment tip, wherein the treatment tip comprises a lumen, is connected to the handle, and is of cylindrical shape; a motor apparatus, wherein the motor apparatus is embedded in the handle, and a power-output shaft of the motor apparatus is arranged in the lumen of the treatment tip; a transducer for focusing ultrasound, wherein the transducer is placed inside the lumen of the treatment tip, and an annular spherical surface of the transducer is directly faced against a wall of the lumen of the treatment tip; a joining beam, wherein two ends of the joining beam are fittingly connected to the power-output shaft of the motor apparatus and the transducer respectively; and the transducer is configured to be driven by the motor apparatus to rotate in a direction perpendicular to, or to move back and forth along, an axial direction of the joining beam.

Preferably, the motor apparatus comprises a first motor and a second motor, wherein: the power-out shaft of the first motor and the second motor are fittingly connected with the joining beam respectively; and the first motor and the second motor are respectively configured to drive the transducer to rotate in a direction perpendicular to, or to move back and forth along, the axial direction of the joining beam.

The vagina ultrasound treatment device as disclosed herein has the following advantages: first, the use of ultrasonic therapy can effectively avoid epidermis damage in the course of treatment, thereby greatly reducing the risk of infection; second, the treatment can be done through circular rotation movement of the transducer in the treatment tip, its linear movement, and/or combination of its circular and liner movement, differentiating from existing technologies which require fixed and simple treatment, thereby speeding up the treatment, decreasing fatigue symptoms of treated patients, and ensuring safety and reliability of the whole course of treatment.

### BRIEF DESCRIPTION OF DRAWINGS

FIG. 1 is a schematic diagram of an ultrasound treatment device according to some embodiments.

FIG. 2 is a structure diagram of a treatment handpiece of an ultrasound treatment device according to some embodiments.

FIG. 3 illustrates an effect of SMAS layer treatment by a transducer according to some embodiments.

FIG. 4 illustrates an effect of dermis treatment by a transducer according to some embodiments.

FIG. 5 illustrates a treatment handpiece with an ultrasound coupling unit according to some embodiments.

FIG. 6 illustrates a treatment handpiece of ultrasound treatment device according to some other embodiments.

FIG. 7 shows a structural schematic diagram of a treatment handpiece for use in ultrasound vagina treatment devices according to a first embodiment.

FIG. 8 shows a structural schematic diagram of a treatment handpiece for use in ultrasound vagina treatment devices according to a second embodiment.

FIG. 9 shows a structural schematic diagram of a treatment handpiece for use in ultrasound vagina treatment devices according to a third embodiment.

FIG. 10 shows the skin structural illustration of vagina tissues.

### DETAILED DESCRIPTION

The following contents explain the implementation methods through specific cases. Those of ordinary skills in the art can easily appreciate other advantages and functionalities of various embodiments through this disclosure. Some embodiments can also be implemented or applied through other different implementation methods. Details in this disclosure can be modified or changed based on different opinions and applications without departing from the scope of the disclosure. It should also be noted that the following implementations and implementation characters can be combined with each other if they do not conflict with each other.

It's important to note that the diagrams of the following implementation examples only illustrate some concepts of various embodiments, and that the figures only illustrate the components related to some embodiments, but are not drawn according to the actual quantity, shape and size of the components. The actual shape, quantity and scale of each component may be altered, and the layout and structure may be more complicated.

Currently majority of the home-use beauty medical devices are physical therapy-standard devices, and compared with medical-standard peer devices, these physical therapy-standard devices have a quite poor effectiveness. In the present market, the medical-standard cosmetic devices include, for example, those used for dewrinkling, hair removal, and hair rejuvenation, etc. So far, however, there are yet no professional-level tightening and lifting home-use devices on the market.

The vagina rejuvenation treatment is one such example. Aging of vagina can cause many problems among women, such as gynecological diseases caused by decreased immunological competence, and vaginal relaxation-induced reduction of quality of life. Especially in postpartum women, they usually has symptoms of vaginal relaxation and aging. Currently the vagina rejuvenation treatment has been implemented in many plastic surgery hospitals. Because of the privacy concerns, most women wouldn't like to be treated in hospitals. Additionally, the surgery has certain risks, and the vagina tightening procedure also has some side effects. Because surgery requires very high expertise, and the surgical view is very small; the surrounding organs, such as bladder, urethra, rectum may be easily damaged, which may result in complications.

Currently high water absorption intense laser is commonly used in the vagina rejuvenation treatment, the mechanism of which is that after absorbing fractional laser by vaginal mucosa, high temperature will be produced instantaneously and some micro gasify holes will be formed to achieve the effect of lifting skin through the healing and regeneration of skin tissue. This treatment method usually generates wounds to the mucosa, which increases the difficulty for recovery and also increases the risk of infection.

There are other methods, such as treating with ultrasound light wave. The principle is that treatment is achieved through recovery and regeneration mechanisms after thermal denaturation of collagen in the skin tissue. The skin structure of vagina tissue mainly comprises mucosa, lamina propria, muscle layer and adventitia. The first layer is a mucosa tissue which comprises a type of keratinized multilayer squamous epithelium forming numerous transverse folds or wrinkles. The mucosa mainly comprises epithelial cell layer with a thickness of about 0.15-0.30 mm. The lamina propria has a thickness of about 2-3 mm, and comprises thin loosen connective tissues, spongy connective tissues and is rich in blood vessels and lymphatic vessels, nerves and elastic fibers; this layer produces liquid in the process of sexual excitement. Below lamina propria are muscle layer and adventitia, wherein the adventitia attaches with muscle layer, and comprises elastin, collagen, lymphatic vessels, nerves, blood vessels etc. During non-sexual excitement, the vagina is about 6.5-7 cm long at the front vagina wall and about 9 cm at the back vagina wall; and during sexual excitement, the vagina is slighted stretched.

The treatment target zone is lamina propria, located at about 0.30-3.5 mm under the skin. Treatment of lamina propria can improve the flexibility of vagina wall and the humidity of vagina. Currently ultrasound treatment on the superficial tissue of the skin is often used in face skin rejuvenation, specifically used in surrounding skin tightening, in a mechanism by heating and denaturing collagen and thermally stimulating regeneration at 65° C.-75° C. The vagina rejuvenation treatment also applies the same mechanism, by thermally stimulating regeneration of the collagen in the lamina propria.

In current laser treatment technology that typically uses 2940 nm laser and 10600 nm laser, vagina rejuvenation is mainly achieved through gasification of vagina mucosa and skin regeneration. After treatment, the wounds generated may increase risk for infection and bleeding after treatment. Because of these wounds, a patient may need 1-7 days off-work after treatment which may have some unwanted influences on life.

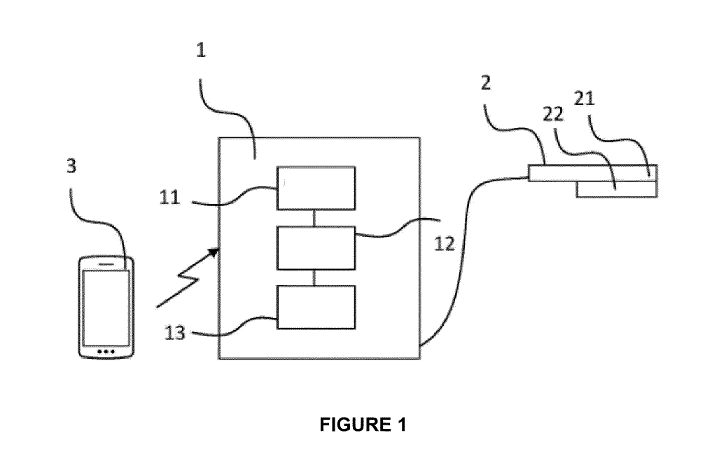

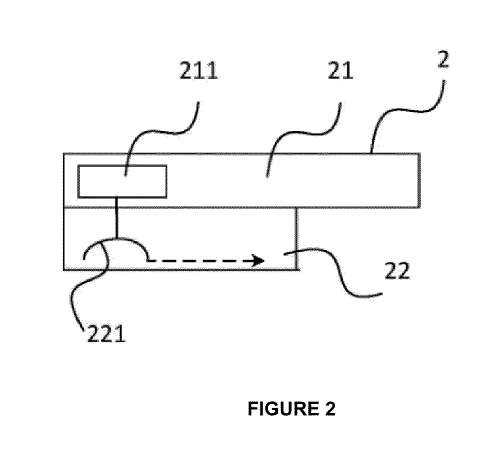

Various embodiments disclosed herein empmloy intensity focus ultrasound (IFUS), also known as micro focused ultrasound (MFUS) to achieve tighten treatment. FIG. 1 shows the schematic diagram of an ultrasound treatment device according to some embodiments. FIG. 2 shows the structure diagram of a treatment handpiece 2 of the ultrasound treatment device according to some embodiments. As show in these figures, the ultrasound treatment device, comprises a treatment main body 1, a treatment handpiece 2 and a display control console 3, wherein: the treatment main body 1 is fittingly connected to the treatment handpiece 2, the display control console 3 is coupled to the treatment main body 1 via an interactive connection; the treatment handpiece 2 comprises a handle 21 and a treatment tip 22, wherein the treatment tip 22 and the handle 21 are connected via a detachable connection, A transducer 221 for focusing ultrasound is arranged in the treatment tip 22, and a motor 211 for driving the transducer 221 to move is arranged in the handle 21; the treatment main body 1 comprises a driving unit 13, a control unit 12 and an ultrasound power unit 11, wherein the driving unit 13 is coupled to the motor 211 in the handle 21, the ultrasound power unit 11 is coupled to the transducer 221 in the treatment tip 22, and the control unit 12 is respectively coupled to the driving unit 13 and the ultrasound power unit 11.

Specifically, in some embodiments, the display control console 3 and the treatment main body 1 are separate from each other, and can exchange data via a wireless connection such as Wi-Fi or blue tooth. The display control console 3 is mainly used to control the inputs and/or outputs of the equipment. For a home-use medical device disclosed herein, to further reduce the cost, a tablet PC or a smart mobile phone may be used as a display control console 3, i.e., through an existing handhold mobile terminal to input and/or output data.

Since the transducer 221 for focusing ultrasound has limited life, in order to reduce user cost, the treatment handpiece 2 is designed to comprise a treatment tip 22 and a handle 21, connected via a detachable connection. The treatment tip 22 is replaceable, which is connected to the handle 21 through a card slot, or a turning knob etc.

In addition, the ultrasound emitted by the transducer 221 typically focuses at a focal point under the skin, forming a focused ultrasound at the specific point during treatment, thus the transducer 221 needs to move in order to treat different sites under the skin. There are multiple methods to move the transducer 221, for example, by moving the whole treatment handpiece 2, or by moving the transducer 221 in the treatment tip 22. In order to achieve more convenient treatment, the approach of moving the transducer 221 in the treatment tip 22 may be applied in some preferred embodiments. Specifically, the transducer 221 in the treatment tip 22 is fittingly connected to the motor 211 in handle 21, and the motor 211 is configured to drive the transducer 221 to move back and forth along the direction of the handle 21, to thereby achieve the movement of transducer 221.

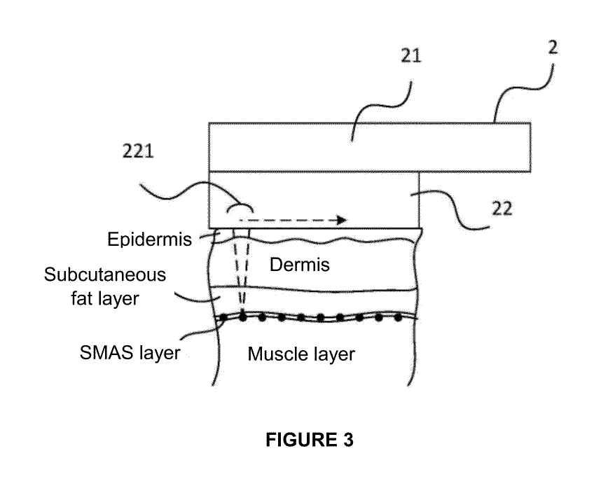

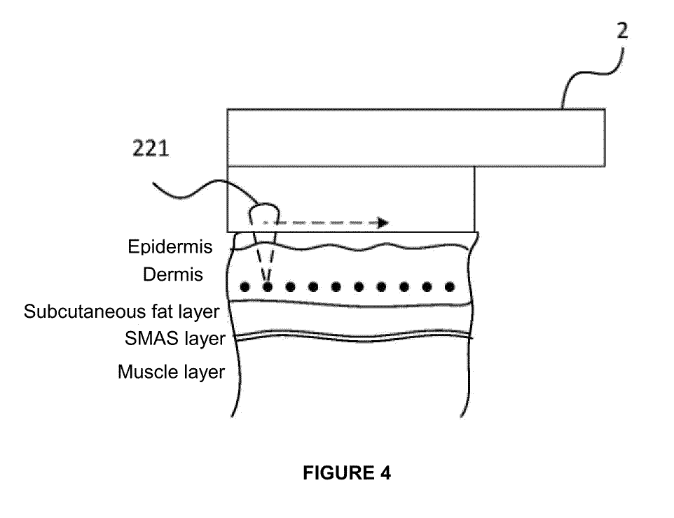

Furthermore, the treatment principle is to treat different layers of tissues under the skin by adjusting the different focus depths of the transducer 221. In one example, if the focus depth is 6 mm and the installation depth is reserved as 1.5 mm, the ultrasonic treatment device can be used to treat tissues 4.5 mm deep under the skin. In another example, FIG. 3 shows a transducer 221 configured for SMAS treatment, where the focus depth is 4.5 mm and the reserved installation depth is 1.5 mm, the treatment depth is 3 mm. In yet another example, FIG. 4 shows a transducer 221 configured for dermis treatment, where the reserved installation depth can vary depending upon different structural designs and can have varying values ranging from 1 mm-5 mm. Meanwhile, the designs can have a setting treatment depth of 4.5 mm for the SMAS layer, or of 3 mm for dermis, or of 1.5 mm for dermis.

To satisfy needs for treatment of different layers, different transducers may be needed, switchable by using different treatment tips 22 or different treatment handpieces.

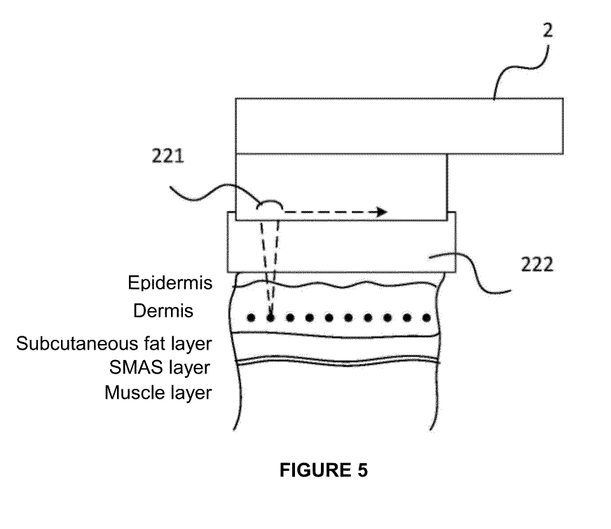

To further reduce the cost, an ultrasound coupling unit 222, such as an ultrasound coupling pad or an ultrasound couplant, may be employed according to some embodiments. To be specific, the ultrasound coupling unit 222 may be disposed between skin and the treatment tip 22, in order to reduce the focus depth under the skin. Using a design and a setting as illustrated in FIG. 3, where the focus depth is 6 mm and the installation depth is 1.5 mm, an ultrasound coupling unit 222 of 1.5 mm height can be used to reach a treatment depth of 3 mm, as illustrated in FIG. 5. The ultrasound couplant requires the use of materials capable of conducting ultrasounds with a conductivity factor greater than about 70%, which can be artificial skin or a solid ultrasonic coupling agent, etc.

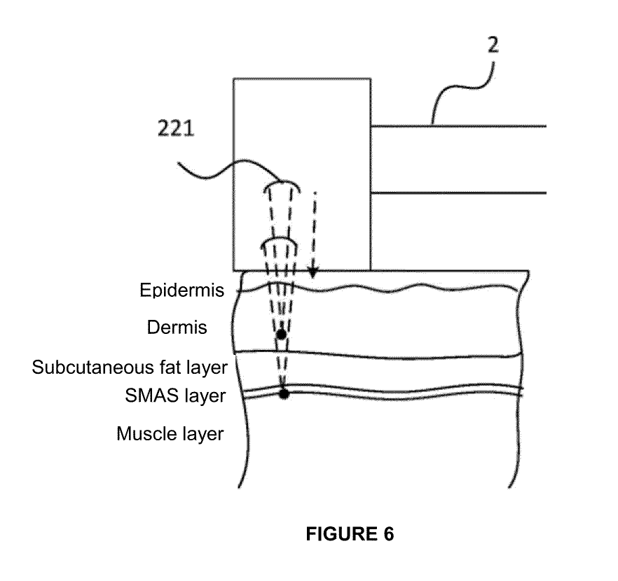

Furthermore, the treatment handpiece 2 can be designed as a single-point treatment handpiece 2 having multiple treatment depths. The transducer 221 in the treatment tip 22 can move vertically to adjust the depth of 1 mm-5 mm, as illustrated in FIG. 6.

Therefore, various embodiments disclosed herein realize home-use applications of focused ultrasound, which fills in the niche of home-use tightening and lifting devices in the market, allowing a medical-standard device to be used in homes, greatly reducing the beauty cost of customers. Meanwhile, the device can be controlled by smart mobile phones or tablet PCs, further lowering the home-use cost. Embodiments disclosed herein effectively overcome many shortcomings in existing technologies, and thus has a high commercial value.

Three embodiments of treatment handpiece for use in an ultrasound vagina treatment device are disclosed herein.

### Embodiment 1

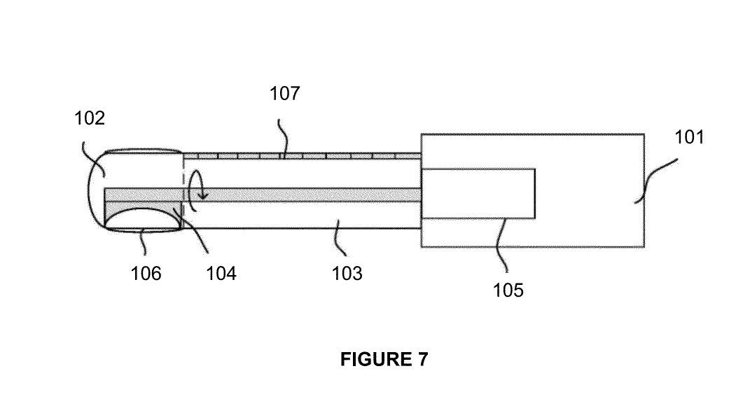

FIG. 7 shows structural schematic diagram of an ultrasound vagina treatment device according to some embodiments, wherein the treatment handpiece is connected with the ultrasound vagina treatment device. In FIG. 7, we can see the treatment handpiece for use in ultrasound devices comprises: a handle 101, a treatment tip with a lumen 102 wherein the treatment tip is connected to the handle 101 and is of a cylindrical shape; a motor 105, wherein the motor 105 is embedded in the handle 101, and a power-output shaft of the motor 105 is arranged in the lumen of the treatment tip 102; a transducer 104 for focusing ultrasound, wherein the transducer 104 is placed in the lumen of the treatment tip 102, and an annular spherical surface of the transducer 104 is directly faced against a wall of the lumen of the treatment tip 102; a joining beam 103, wherein the two ends of the joining beam 103 are fittingly connected to the power-output shaft of the motor 105 and the transducer 104 respectively, and the transducer 104 is configured to be driven by the motor 105 to rotate in a direction perpendicular to the axial direction of the joining beam 103.

Because of the characteristics of the focused ultrasound, energy can be focused on only one point during one single treatment, and thus treatment of multiple different areas requires moving transducer. The treatment handpiece for use in ultrasound vagina treatment devices as disclosed herein rotates the transducer 104 to treat a section perpendicular to the treatment tip 102. Treatment of this section is thus achieved by rotating the transducer 104. The transducer 104 is configured to connect to the joining beam 103, the motor 105 is arranged inside the treatment handpiece, and the motor 105 drives the joining beam 103 to rotate, thereby achieving a faster treatment speed and a better efficiency compared with existing single point treatment.

The usual vaginal wall is 20-30 mm in diameter. Thus in order to ensure good contact with vaginal wall, a protrusion on the treatment area is arranged, wherein the diameter of the treatment handpiece may be 25-35 mm, and the diameter of the protrusion 106 is 2-5 mm bigger than that of the treatment handpiece. Specifically in one example, the diameter of the protrusion 106 is 35 mm, the diameter of other parts of the treatment handpiece is 30 mm; this configuration can ensure well contact between vaginal wall and treatment handpiece. As shown in FIG. 7, a annular protrusion 106 is arranged around the treatment tip 102, and the protrusion 106 is arranged to directly face the annular spherical surface of the transducer 104, and to circle around the peripheral surface of the transducer 104.

Furthermore, for example, in a treatment tip 102 with a diameter of 35 mm, the length of the annular vaginal wall covered by the treatment tip 102 is π*2R=110 mm, set the focal distance as 2 mm, then 55 treatment points are needed, in other words, the moving angle of the transducer 104 is 6.6° each treatment point. In some situation, the moving angle can be changed in a range of 3-30° to achieve the entire vagina wall treatment. In some other situation, cyclic treatment angle can be adjusted, for example, to be set as 30°-330° and also to reserve 60° as untreated. Specifically this setting can be used for treatment in orificium vaginae, where the skin tissue between vaginal wall and urethra wall is relatively thin, which can avoid the treatment-induced side effects, such as burning urine etc.

After the entire cyclic treatment is finished, the treatment handpiece needs to be moved backwards to treat other section points. There are scale marks 107 arranged on the treatment tip 102 in order to mark the movement. For example, the scale is 10 cm for the first treatment and 9.5 cm for the second treatment, etc. Specifically, a set of scale marks 107 are arranged on an outer surface of, and along an axial direction of, the joining beam

In addition, the design of the treatment handpiece as a combination of a treatment tip 102 and a handle can avoid the risk of cross-infection, and the treatment tip 102 is also designed as consumable. Since vagina is body cavity, to ensure a better effect, the entire vagina needs to undergo 360° treatment, and for this reason, the treatment tip 102 and joining beam 103 both take a cylindrical or annular structure, which can carry out 360° treatment of the entire vagina.

### Embodiment 2

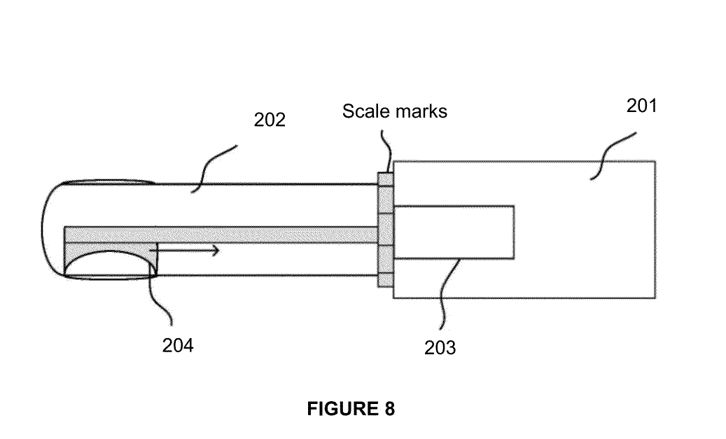

Additionally, FIG. 8 shows a structural schematic diagram of ultrasound vagina treatment device according to a second embodiment. As illustrated in FIG. 8, the treatment handpiece comprises: a handle 201, a treatment tip with lumen 202, wherein the treatment tip is connected to the handle 201, and is of a cylindrical shape; a motor 205, wherein the motor 205 is embedded in the handle 201, and a power-output shaft of the motor 205 is arranged in the lumen of the treatment tip 202; a transducer 204 for focusing ultrasound, wherein the transducer 204 is placed inside the lumen of the treatment tip 202 and is connected with the power-output shaft of the motor 205, and an annular spherical surface of the transducer 204 is directly faced against a wall of the lumen of the treatment tip 202, and the transducer 204 is configured to be driven by the motor 205 to move back and forth along an axial direction of the treatment tip 202.

In this embodiment, the transducer 204 can move in a vertical direction of the vaginal wall, for example, move from inside to outside. After the transducer finishes one vertical movement relative to the vaginal wall, it needs only to rotate to change the treatment directions, thereby greatly reducing the operation time and improving the treatment efficiency due to the feature that a therapist only needs to rotate treatment direction multiple times.

In one example, the transducer 204 is fixed on the joining beam 103, and can move in the same straight line. Where the specific treatment length is set as 5-15 cm, the point interval can be 0.5-3 mm. For example, where the treatment length is set as 10 cm, the point interval can be 2 mm, then focusing at 50 focal points on a line is necessary. Along the 10 cm treatment length, we can set actual length for firing ultrasound focusing, such as 2 cm-8 cm as the treatment zone. In this way, the vagina front wall and back wall do not be treated at the same time, and the vagina and urethra junction can also be selectively untreated.

When treatment in the vertical direction is finished, we need to rotate the treatment tip 202, to treat another line on the vagina wall. A set of scale marks are arranged on the turning knob, and can have 10-50 scale marks. When treatment of one line is finished, then the treatment tip 202 can be turned to treat another line.

### Embodiment 3

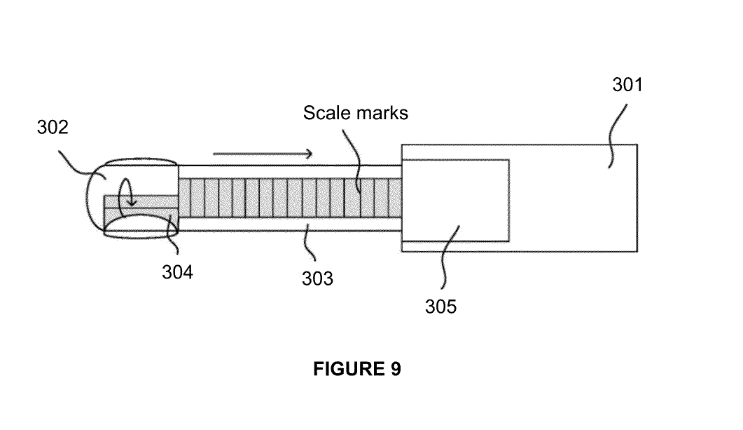

Another type of treatment handpiece for use in ultrasound vagina treatment devices can be provided. FIG. 9 shows structural schematic diagram of treatment handpiece of vagina treatment device according to a third embodiment. From FIG. 9, we can see that the treatment handpiece comprises: a handle 301; a treatment tip with lumen 302, wherein the treatment tip 302 is connected to the handle 301, and is of a cylindrical shape; a motor apparatus 305, wherein the motor apparatus is embedded in the handle 301, and a power-output shaft of the motor apparatus 305 is arranged in the lumen of the treatment tip 302; a transducer 304 for focusing ultrasound, wherein the transducer is placed inside the lumen of the treatment tip 302, and an annular spherical surface of the transducer 304 is directly faced against a wall of the lumen of the treatment tip 302; a joining beam 303, wherein the two ends of the joining beam are fittingly connected to the power-output shaft of the motor apparatus 305 and the transducer 304 respectively, and the transducer 304 is configured to be driven by the motor apparatus 305 to rotate in a direction perpendicular to, or to move back and forth along, the axial direction of the joining beam 303.

The above mentioned embodiment 3 is different form embodiment 1 and embodiment 2 in that the transducer 304 first makes circular movement inside the treatment tip 302, and then the treatment tip 302 moves backwards to do the treatment at the next section. Specifically, the treatment tip 302 can perform 360° rotation treatment, and after the circular treatment is finished, the treatment tip is electrically driven to move backwards for the treatment of other section points.

Specifically, the two movement directions can be controlled by the motor apparatus 305 that comprises two motors, one to control linear movement, and the other to control circular movement. In a specific embodiment, the motor apparatus 305 comprises two motors, a first motor and a second motor, each comprising a power-out shaft, wherein the power-out shaft of the first motor and the power-out shaft of the second motor are respectively fittingly connected with the joining beam 303, and the first motor and the second motor are respectively configured to drive the transducer 304 to rotate in a direction perpendicular to, or to move back and forth along, the axial direction of the joining beam 303.

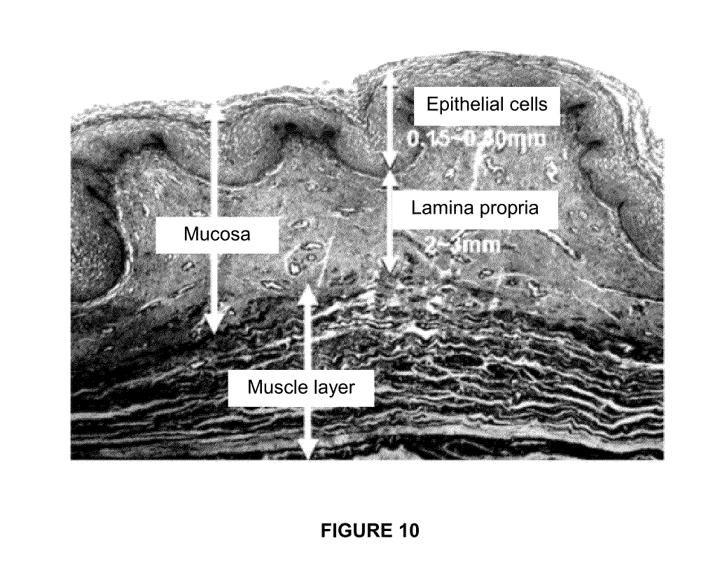

Disclosed herein is a kind of treatment handpiece for use in ultrasound wave vagina treatment devices, which can be used with treatment main body of the ultrasound treatment devices to perform treatment to rejuvenate vagina. Specifically, the effects of vagina treatment are illustrated in FIG. 10.

As FIG. 10 shows, the skin structure of vagina tissue mainly comprises mucosa, lamina propria, muscle layer and adventitia. The first layer is a mucosa tissue, which comprises a type of keratinized multilayer squamous epithelium forming numerous transverse folds or wrinkles. The mucosa mainly comprises epithelial cell layer with a thickness of about 0.15-0.30 mm. The lamina propria has a thickness of about 2-3 mm, and comprises thin loosen connective tissues, spongy connective tissues and is rich in blood vessels and lymphatic vessels, nerves and elastic fibers; this layer produces liquid in the process of sexual excitement. Below lamina propria are muscle layer and adventitia, wherein the adventitia attaches with muscle layer and comprises elastin, collagen, lymphatic vessels, nerves, blood vessels etc. During non-sexual excitement, the vagina is about 6.5-7 cm long at the front vagina wall and about 9 cm at the back vagina wall; and during sexual excitement, the vagina is slighted stretched.

The treatment target zone is lamina propria, located at about 0.30-3.5 mm under the skin. Treatment of lamina propria can improve the flexibility of vagina wall and the humidity of vagina. Currently ultrasound treatment on the superficial tissue of the skin is often used in face skin rejuvenation, specifically used in surrounding skin tightening, in a mechanism by heating and denaturing collagen and thermally stimulating regeneration at 65° C.-75° C. The vagina rejuvenation treatment also uses the same mechanism, by thermally stimulating regeneration of the collagen in the lamina propria.

To sum up, vagina tightening is a new field of application. Embodiments disclosed herein are different from existing technologies, such as weak-current treatment, which has low efficiency, and laser treatment, which carries high risks. It uses new energy source for the treatment of vagina, and has an improved treatment effect and reduced risk of side effects.

In addition, treatment can be done through circular rotation movement of the transducer in the treatment tip, its liner movement, and/or combination of its circular and liner movement, differentiating existing technologies which typically require fixed and simple treatment, thereby speeding up the treatment, decreasing fatigue symptoms of treated patients, and ensuring safety and reliability of the whole course of treatment. Therefore, various embodiments disclosed herein effectively overcome the shortcomings of existing technologies, and has high industry utilization value.

These above mentioned implementation examples are for illustration of the principle and effects of some embodiments only and are not limiting. Any modifications or changes, made by people of ordinary skills in the field, on top of this disclosure, so long as they do not depart from the spirit and scope of this disclosure, are still covered by the claims in this disclosure.

1. A ultrasonic treatment device, comprising:
a treatment main body,
a treatment handpiece, and
a di splay control console;
wherein:
the treatment main body is fittingly connected to the treatment handpiece;
the display control console is coupled to the treatment main body via an interactive connection;
the treatment handpiece comprises a handle and a treatment tip, wherein the handle and the treatment tip are connected via a detachable connection, a transducer for focusing ultrasound is arranged in the treatment tip, and a motor for driving the transducer to move is arranged in the handle;
the treatment main body comprises a driving unit, a control unit and a ultrasound power unit, wherein the driving unit is coupled to the motor in the handle, the ultrasound power unit is coupled to the transducer in the treatment tip, and the control unit is respectively coupled to the driving unit and the ultrasound power unit; and
the detachable connection via which the handle and the treatment tip are connected is through a card slot or a turning knob.

2. The ultrasonic treatment device according to claim 1, wherein in the treatment handpiece, the transducer in the treatment tip is fittingly connected to the motor in the handle, and the motor is configured to move the transducer along a vertical direction or a horizontal direction.

3. The ultrasonic treatment device according to claim 1, further comprising an ultrasound coupling unit configured for adjusting treatment depth, wherein the ultrasound coupling unit is arranged on a treatment side of the treatment tip.

4. The ultrasonic treatment device according to claim 3, wherein the ultrasound coupling unit comprises an ultrasound coupling pad or an ultrasound couplant, wherein the ultrasound couplant comprises a material capable of conducting ultrasounds with a conductivity factor greater than about 70%.

5. The ultrasonic treatment device according to claim 4, wherein the ultrasound couplant comprises at least one of artificial skin and a solid ultrasonic coupling agent.

6. A treatment handpiece for use in an ultrasound vagina treatment device, comprising:
a handle,
a treatment tip,
a transducer for focusing ultrasound, and
a driving means;
wherein:
the treatment tip comprises a lumen, is connected to the handle, and is of a cylindrical shape;
the transducer is arranged in the lumen of the treatment tip such that an annular spherical surface of the transducer is directly faced against a wall of the lumen of the treatment tip; and
the driving means is configured to drive the transducer to move in the lumen of the treatment tip.

7. The treatment handpiece according to claim 6, wherein the driving means comprises a motor and a joining beam, wherein:
the motor is arranged in the handle, and a power-output shaft of the motor extends in the lumen of the treatment tip; two ends of the joining beam are fittingly connected to the transducer and the power-output shaft of the motor respectively; and the motor is configured to drive the transducer to rotate in a direction perpendicular to an axial direction of the joining beam.

8. The treatment handpiece according to claim 7, wherein an outer surface of the treatment tip has a protrusion clinging to the treatment tip, and the protrusion is directly faced against the annular spherical surface of transducer.

9. The treatment handpiece according to claim 7, wherein a set of scale marks are arranged on an outer surface of, and along an axial direction of, the joining beam.

10. The treatment handpiece according to claim 7, wherein the transducer is configured to rotate at an angle ranging from 3 to 30°.

11. The treatment handpiece according to claim 7, wherein a diameter of the cylindrical shape of the treatment tip is 25-35 mm.

12. The treatment handpiece according to claim 6, wherein the driving means comprises a motor, wherein:
the motor is arranged in the handle, and a power-output shaft of the motor extends in the lumen of the treatment tip; the transducer is connected with the power-output shaft of the motor; and the motor is configured to drive the transducer to move back and forth along an axial direction of the treatment tip.

13. The treatment handpiece according to claim 12, wherein a linear distance of the transducer configured to move back and forth along the axial direction of the treatment tip is about 0.5-5 mm.

14. The treatment handpiece according to claim 12, wherein a set of scale marks are arranged on an outer surface of the treatment tip at a connection between the treatment tip and the handle.

15. The treatment handpiece according to claim 6, wherein the driving means comprises a motor apparatus and a joining beam, wherein:
the motor apparatus is arranged in the handle, and a power-output shaft of the motor apparatus extends in the lumen of the treatment tip; two ends of the joining beam are fittingly connected to the transducer and the power-output shaft of the motor apparatus respectively; and the motor apparatus is configured to drive the transducer to rotate in a direction perpendicular to, and to move back and forth along, an axial direction of the joining beam.

16. The treatment handpiece according to claim 15, wherein the motor apparatus comprises a first motor and a second motor, and the power-output shaft of the motor apparatus comprises a first power-output shaft and a second power-output shaft, wherein:
the first power-out shaft extends from the first motor; the second power-out shaft extends from the second motor; and the first power-output shaft and the second power-output shaft are fittingly connected with the joining beam respectively; and
the first motor and the second motor are respectively configured to drive the transducer to rotate in a direction perpendicular to, and to move back and forth along, the axial direction of the joining beam.
